(19) United States
(12) Patent Application Publication
(10) Pub. No.: US 2018/0028219 A1
(43) Pub. Date: Feb. 1, 2018

(54) POLYPECTOMY SNARE DEVICES

(71) Applicant: BOSTON SCIENTIFIC SCIMED, INC., Maple Grove, MN (US)

(72) Inventors: MARTYN G. FOLAN, Loughrea (IE); MATTHEW MONTAGUE, Oranmore (IE); DAMIEN V. NOLAN, Craughwell (IE); ENDA CONNAUGHTON, Craughwell (IE); THOMAS M. KEATING, Tuam (IE); MARTIN HYNES, Galway (IE)

(21) Appl. No.: 15/661,720

(22) Filed: Jul. 27, 2017

Related U.S. Application Data
(60) Provisional application No. 62/367,922, filed on Jul. 28, 2016.

(51) Int. Cl. A61B 17/3205; A61B 18/14

(52) U.S. Cl. CPC A61B 17/32056; A61B 18/1485; A61B 2017/320064

(57) ABSTRACT

Polypectomy devices and methods for making and using polypectomy devices are disclosed. An example polypectomy device may include an elongate sheath having a proximal end and a distal end. A handle assembly may be coupled to the proximal end. The handle assembly may include a first actuator and a second actuator. A first shaft may extend through the sheath and may be coupled to the first actuator. An end effector may be coupled to the first shaft. A second shaft may extend through the sheath and may be coupled to the second actuator. A retention member may be coupled to the second shaft. The first actuator may be designed to shift the end effector between a housed configuration and a deployed configuration. The second actuator may be designed to shift the retention member between a retracted configuration and a holding configuration.

### CROSS-REFERENCE TO RELATED APPLICATIONS

This application claims priority under 35 U.S.C. § 119 to U.S. Provisional Application Ser. No. 62/367,922, filed Jul. 28, 2016, the entirety of which is incorporated herein by reference.

### TECHNICAL FIELD

The present disclosure pertains to medical devices, and methods for manufacturing medical devices. More particularly, the present disclosure pertains to polypectomy devices including an end effector and a retention member.

### BACKGROUND

A wide variety of intracorporeal medical devices have been developed for medical use. Some of these devices include guidewires, catheters, endoscopic devices, biopsy devices, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A polypectomy device is disclosed. The polypectomy device comprises: an elongate sheath having a proximal end and a distal end; a handle assembly coupled to the proximal end, the handle assembly including a first actuator and a second actuator; a first shaft extending through the sheath and coupled to the first actuator; an end effector coupled to the first shaft; a second shaft extending through the sheath and coupled to the second actuator; a retention member coupled to the second shaft; wherein the first actuator is designed to shift the end effector between a housed configuration and a deployed configuration; and wherein the second actuator is designed to shift the retention member between a retracted configuration and a holding configuration.

Alternatively or additionally to any of the embodiments above, the first actuator and the second actuator are independently controllable.

Alternatively or additionally to any of the embodiments above, the end effector includes a snare loop.

Alternatively or additionally to any of the embodiments above, the retention member includes a retention barb.

Alternatively or additionally to any of the embodiments above, the retention member includes a spike.

Alternatively or additionally to any of the embodiments above, the first shaft and the second shaft are arranged substantially parallel to one another within at least a portion of the sheath.

Alternatively or additionally to any of the embodiments above, the sheath includes a distal holding member.

Alternatively or additionally to any of the embodiments above, the distal holding member is positioned along an interior surface of the sheath.

Alternatively or additionally to any of the embodiments above, the distal holding member includes a bristle.

Alternatively or additionally to any of the embodiments above, the distal holding member includes a pair of bristles disposed along opposite sides of the sheath.

A method for removing polyps is disclosed. The method comprises: advancing a polypectomy snare device through a body lumen to a position adjacent to a polyp, the polypectomy snare device comprising: an elongate sheath having a proximal end and a distal end, a handle assembly coupled to the proximal end, the handle assembly including a first handle and a second handle, a first shaft extending through the sheath and coupled to the first handle; a snare loop coupled to the first shaft, a second shaft extending through the sheath and coupled to the second handle, a retention member coupled to the second shaft, wherein the first handle is designed to shift the snare loop between a housed configuration and a deployed configuration, and wherein the second handle is designed to shift the retention member between a retracted configuration and a holding configuration. The method further comprising actuating the first handle such that the snare loop engages the polyp and actuating the second handle such that the retention member engages the polyp; and severing the polyp with the snare loop.

Alternatively or additionally to any of the embodiments above, the first actuator and the second actuator are independently controllable.

Alternatively or additionally to any of the embodiments above, the retention member includes a retention barb.

Alternatively or additionally to any of the embodiments above, further comprising retracting the severed polyp into the sheath.

Alternatively or additionally to any of the embodiments above, the sheath includes one or more bristles positioned along an interior surface of the sheath.

Alternatively or additionally to any of the embodiments above, retracting the severed polyp into the sheath includes engaging the severed polyp with at least one of the one or more bristles.

Alternatively or additionally to any of the embodiments above, further comprising: actuating the first handle so that the snare loop engages a second polyp; actuating the second handle such that the retention member engages the second polyp; and severing the second polyp with the snare loop.

A polypectomy snare device is disclosed. The polypectomy device comprises: an elongate sheath having a proximal end and a distal end; a handle assembly coupled to the proximal end, the handle assembly including a first handle and a second handle; wherein the first handle and the second handle are independently controllable; a first shaft slidably disposed within the sheath and coupled to the first handle; a snare loop coupled to the first shaft; a second shaft slidably disposed within the sheath and coupled to the second handle; a barbed retention member coupled to the second shaft; wherein the first handle is designed to shift the first shaft axially relative to the sheath; and wherein the second handle is designed to shift the second shaft axially relative to the sheath.

Alternatively or additionally to any of the embodiments above, the sheath includes one or more bristles positioned along an interior surface of the sheath.

Alternatively or additionally to any of the embodiments above, the sheath includes a pair of bristles disposed along opposite sides of the sheath.

A medical device suitable for removing one or more polyps is disclosed. The medical device comprises: an elongate sheath having a proximal end and a distal end; a handle assembly coupled to the proximal end, the handle assembly including a first handle and a second handle; a first shaft extending through the sheath and coupled to the first handle; a snare loop coupled to the first shaft; a second shaft extending through the sheath and coupled to the second handle; a retention member coupled to the second shaft; wherein the first handle is designed to shift the snare loop between a housed configuration and a deployed configuration; wherein the first handle is designed to be actuated so that the snare loop can engage a polyp; wherein the second handle is designed to shift the retention member between a retracted configuration and a holding configuration; and wherein the second handle is designed to shift the retention member so that the retention member engages the polyp.

Alternatively or additionally to any of the embodiments above, the first actuator and the second actuator are independently controllable.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device.

FIG. 2 is a side view of an example medical device.

FIG. 3 is a side view of an example medical device.

FIG. 4 is a partial cross-sectional side view of an example medical device disposed within a body lumen.

FIG. 5 is a partial cross-sectional side view of an example medical device disposed within a body lumen.

FIG. 6 is a partial cross-sectional side view of an example medical device disposed within a body lumen.

FIG. 7 is a partial cross-sectional side view of an example medical device disposed within a body lumen.

FIG. 8 is a partial cross-sectional side view of an example medical device disposed within a body lumen.

FIG. 9 is a partial cross-sectional side view of an example medical device.

FIG. 10 is a partial cross-sectional side view of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Colonic polypectomy generally corresponds to the removal of colorectal polyps, for example, in order to prevent them from turning cancerous. Some polypectomy systems may include a snare that is engaged with a polyp. The polyp may be severed with the snare. For a number of reasons, it may be desirable to retain or hold the polyp so that the polyp can be scrutinized and/or otherwise analyzed (e.g., for pathology). Once the poly is severed, however, the polyp could be ejected or otherwise move from the clinician's field of view. It may be desirable to retain the polyp so that the polyp can be suitable analyzed and/or scrutinized.

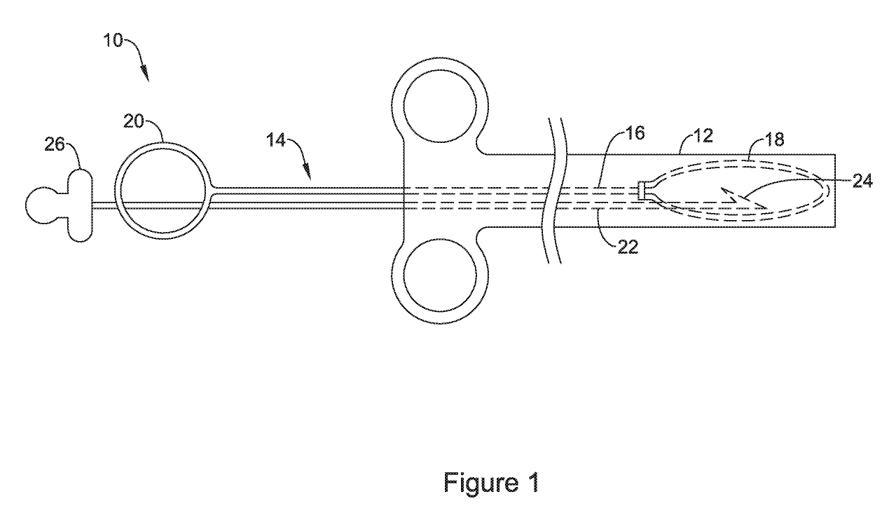

FIG. 1 illustrates an example polypectomy device 10. Device 10, which is shown schematically, may include a shaft or sheath 12. A handle assembly 14 may be coupled to sheath 12. Sheath 12 and handle assembly 14 may have a variety of different forms and/or configurations. For example, sheath 12 may have a length that is suitable to extend through an endoscope to a position within a body lumen. This may include a body lumen along the digestive tract such as along the small intestine and/or colon. Other body lumens may also be accessed with sheath 12. In some instance, a portion of sheath 12 (e.g., a distal portion) may be substantially transparent so that sheath 12 has a reduced impact on visualization of the anatomy and/or target tissue during an intervention. In general, device 10 includes a structural feature for engaging and/or removing tissue as well as a structural feature for retaining and/or holding the tissue. By retaining the tissue, the tissue may be scrutinized, analyzed, and/or removed from the body lumen.

A first shaft 16 may be slidably disposed within sheath 12. An end effector 18 may be coupled to first shaft 16. End effector 18 is generally designed to engage a body tissue such as a polyp. In at least some instances, end effector 18 takes the form of a snare loop. The form of snare loop 18 may vary. For example, snare loop 18 may be formed from a monofilament wire or from a braided wire. Snare loop 18 may include one or more gripping features or “teeth” to aid in grabbing onto target tissue. First shaft 16 may include an electrical connector so that electrical current (e.g., cautery current) can be applied to snare loop 18. In other instances, end effector 18 may include a biopsy forceps, clevis, cutting member or blade, or the like.

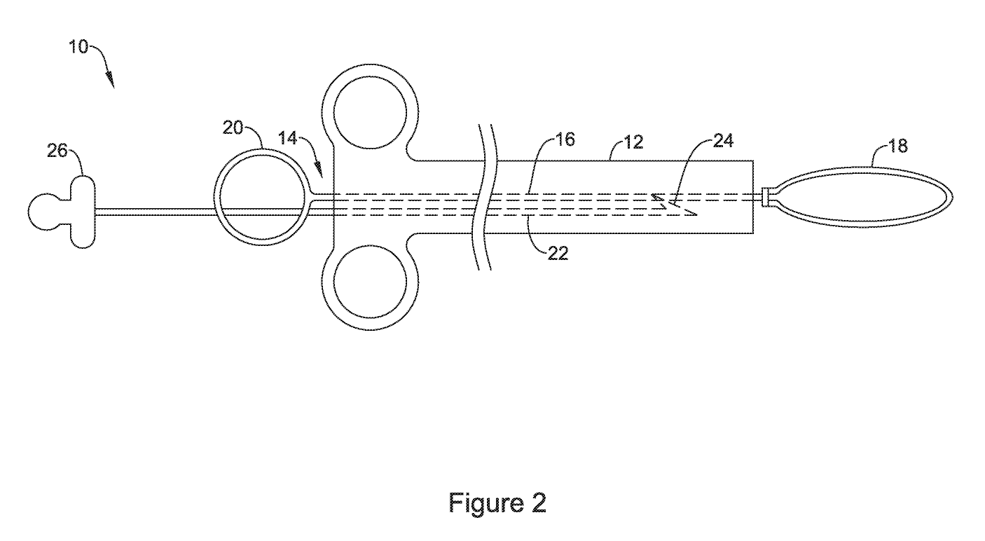

Handle assembly 14 may include a first handle or actuator 20 for moving/sliding first shaft 16 relative to sheath. For example, FIG. 2 illustrates first handle 20 moved to a distal position. When doing so, snare loop 18 may emerge from the distal end of sheath 12. The form of first handle 20 may vary. In some instances, first handle 20 may include a ring or end that can be engaged with the thumb or fingers of a clinician and slid in the proximal direction and/or the distal direction. Other handles are contemplated.

A second shaft 22 may be slidably disposed within sheath 12. A retention member 24 may be coupled to second shaft 22. In at least some instances, retention member 24 takes the form of a barb or spike disposed on the end of second shaft 22. Alternatively, retention member 24 may be a retention structure (e.g., including a barb, spike, needle, or the like) that is attached to second shaft 22. Regardless of the form, retention member is designed to engage and hold on to tissue. When doing so, a clinician may be able to pull tissue back into sheath 12, against the distal end of sheath 12, or otherwise hold the tissue at a location that allows the clinician to visualize the tissue (e.g., via an imaging device associated with or extending through an endoscope). In some instances, a vacuum may be applied through sheath 12 or via a separate vacuum tube to secure the position of the polyp relative to the sheath 12.

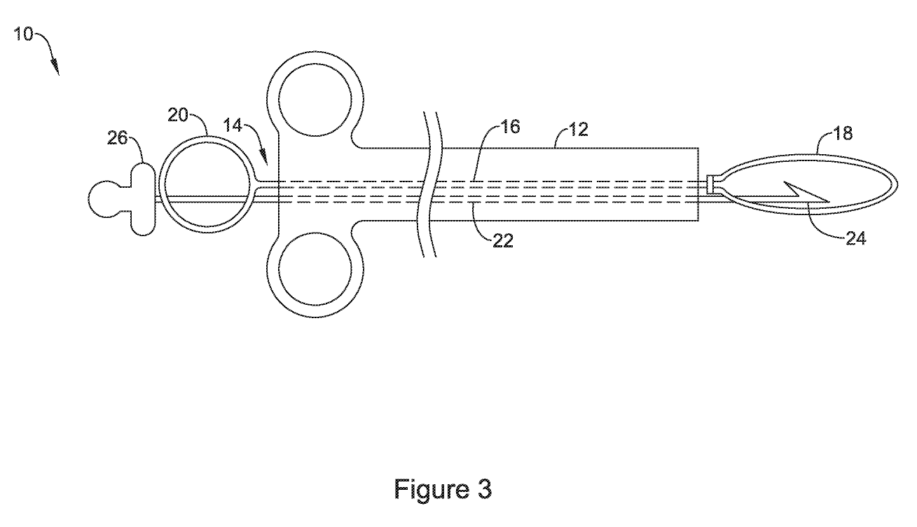

Handle assembly 14 may include a second handle or actuator 26 for moving/sliding shaft 22 relative to sheath. For example, FIG. 3 illustrates second handle 26 moved to a distal position. When doing so, retention member 24 may emerge from the distal end of sheath 12 and be brought into engagement with tissue. By doing so, retention member 24 may extend at least partially into the tissue or through the tissue.

In at least some instances, first handle 20 and second handle 26 can be actuated independently of one another. This may allow a clinician to precisely control the position of snare loop 18 and retention member 24 independently of one another. In some instances, first handle 20 and second handle 26 are independent handles or actuators on handle assembly 14. Alternatively, first handle 20, second handle 26, or both are separate from handle assembly 14 and may be used independently of handle assembly 14. In at least some instances, first handle 20 and second handle 26 extend within sheath 12 in a side-by-side manner along at least a portion of the length of sheath 12. Other arrangements are contemplated.

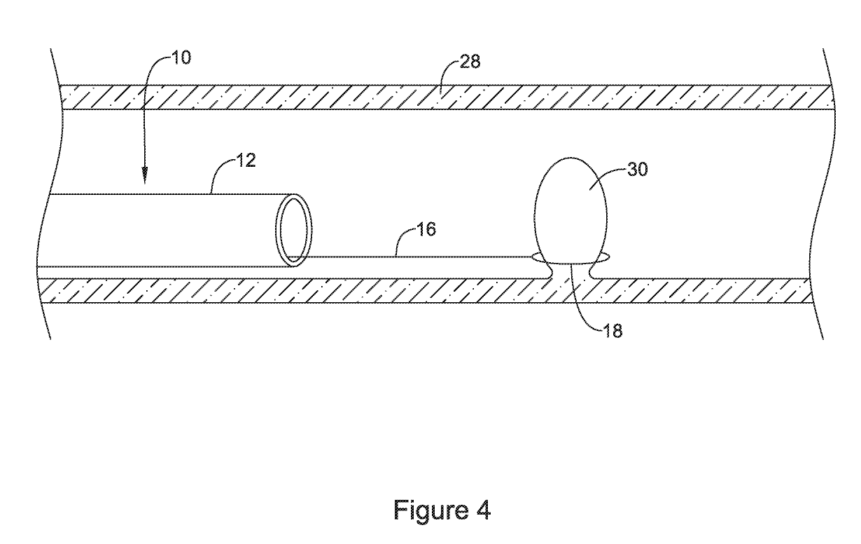

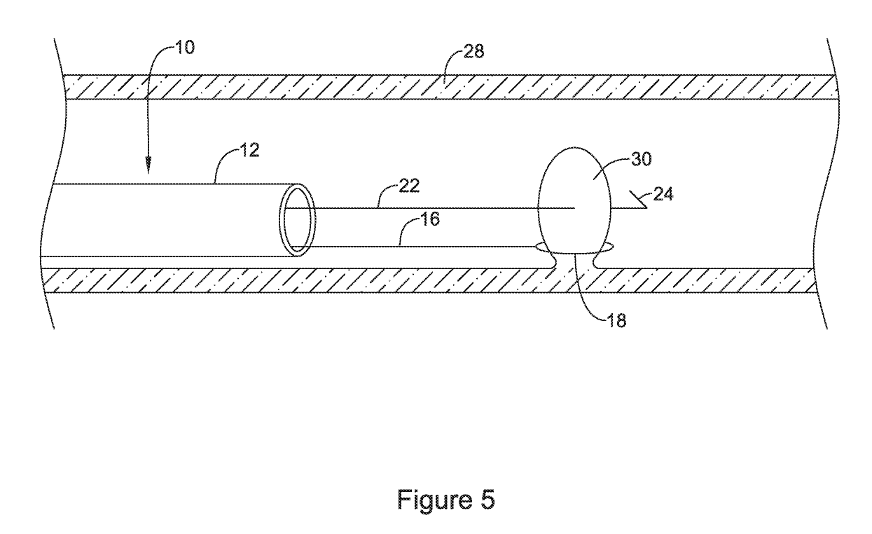

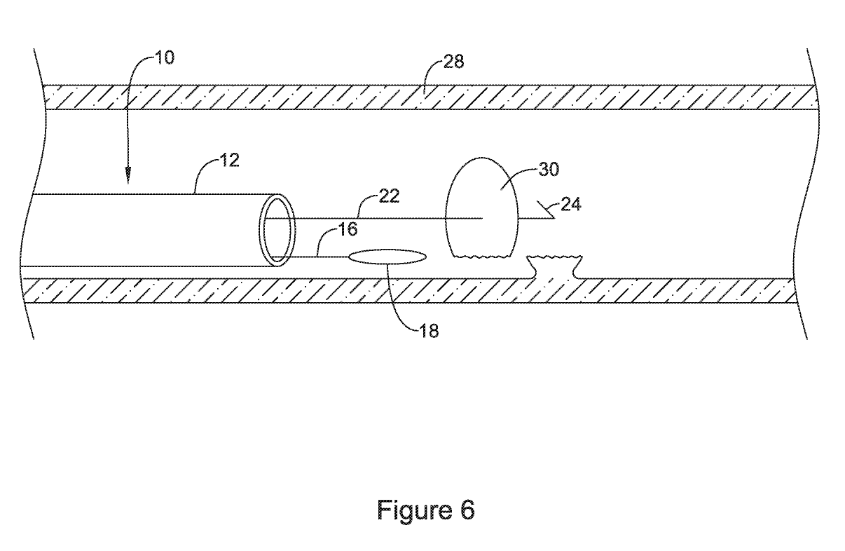

FIGS. 4-8 schematically illustrate the use of device 10. For example, FIG. 4 illustrates device 10 disposed within a body lumen 28. In this example, first handle 20 is actuated so that snare loop 18 can engage a target tissue such as a polyp 30. When snare loop 18 is suitably engaged with polyp 30, second handle 26 may be actuated so that retention member 24 can engage polyp 30 as shown in FIG. 5. When retention member 24 is suitably engaged with polyp 30, first handle 20 can be proximally retracted to sever polyp 30 as shown in FIG. 6. Severing polyp 30 may occur by simply pulling on snare loop 18 in order to simply cut through polyp 30. In some of these and in other embodiments, severing polyp 30 may include pressing the distal end of sheath 12 into engagement with polyp 30 and pulling snare loop 18 through polyp 30 while holding polyp 30 with sheath 12. In some instances, second handle 26 can be retracted along with first handle 20 during the cutting/severing process. However, this is not required.

It may be desirable to pull the severed polyp 30 into sheath 12 after severing polyp 30. For example, second handle 26 may be proximally retracted until polyp 30 engages the distal end of sheath 12, extends partially into sheath 12, and/or extends completely into sheath 12. In some instances, second handle 26 may be used to completely remove polyp 30 from the patient.

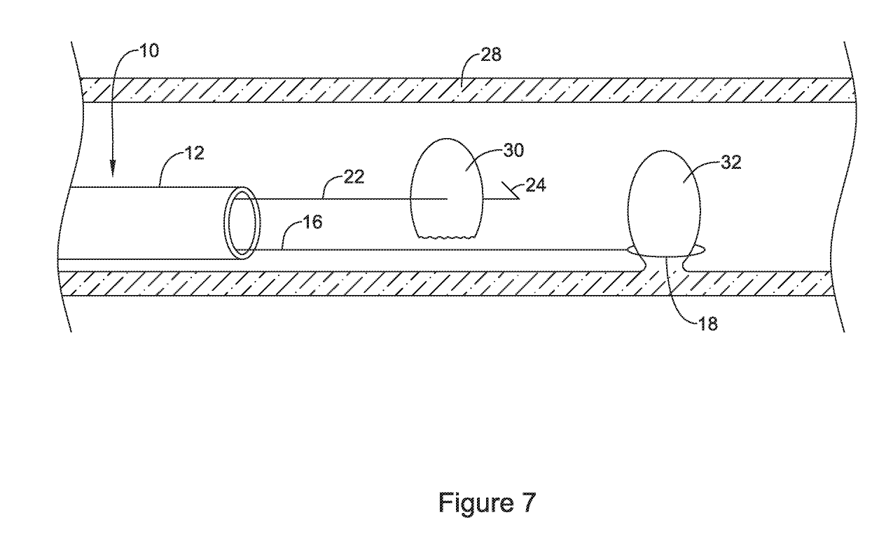

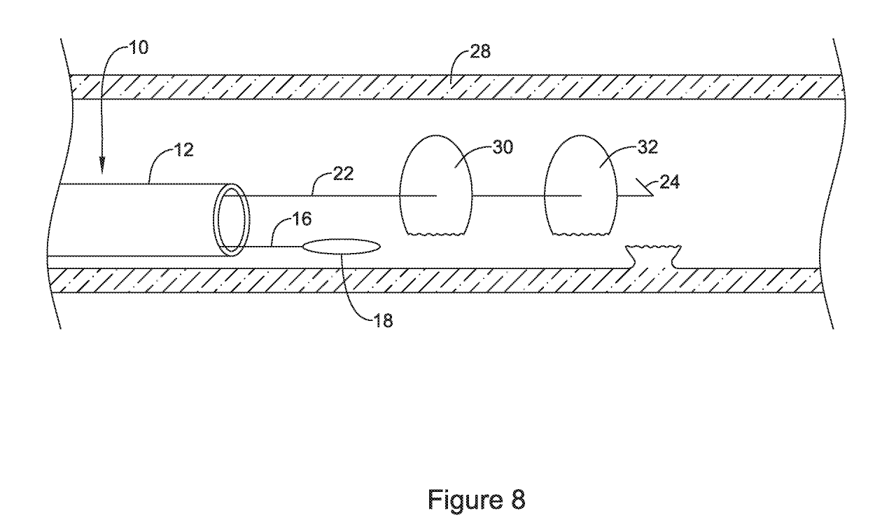

In at least some instances, device 10 may be utilized to remove a plurality of polyps, for example, during a single intervention. For example, FIG. 7 illustrates that with retention member 24 engaged with polyp 30, first handle 20 may be actuated so that snare loop 18 may engage a second polyp 32. Second handle 26 may be actuated so that retention member 24 can engage second polyp 32 and first handle 20 can be proximally retracted to sever second polyp 32 as shown in FIG. 8. The process may be repeated to remove additional polyps.

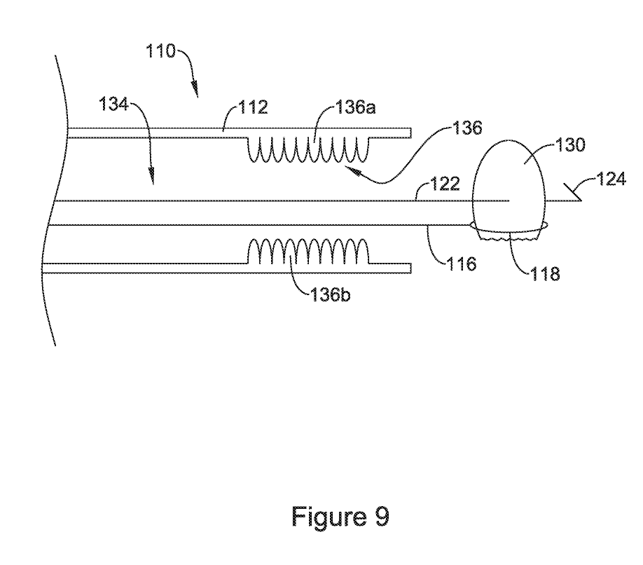

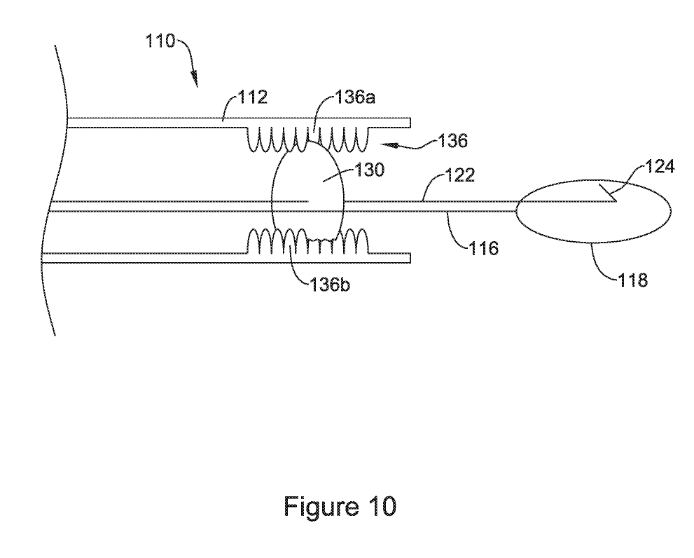

FIGS. 9-10 illustrate another example polypectomy device 110 that may be similar in form and function to other devices disclosed herein. Device 110 includes a sheath 112. A handle assembly (not shown, may be similar to handle assembly 14) may be coupled to sheath 112. First shaft 116 may be slidably disposed within sheath 112. End effector/snare loop 118 may be coupled to first shaft 116. Second shaft 122 may also be slidably disposed within sheath 112. Retention member 124 may be coupled to second shaft 122. While not shown, a first handle and a second handle may be coupled to first shaft 116 and second shaft 122, respectively, and may be used in a manner similar to handles 16/22.

In at least some instances, sheath 112 may include a distal holding member 136. Distal holding member 136 may be designed to hold tissue (e.g., severed polyps such as polyp 130) within the interior of a lumen 134 of sheath 112. In some instances, distal holding member 136 may take the form of one or more bristles (e.g., a first bristle 136a and a second bristle 136b) disposed along the interior of sheath 112. Bristles may have a relatively rough, brush-like structure that can engage and hold polyp 130 within lumen 134 when polyp 130 is retracted into lumen 134 of sheath 112 as shown in FIG. 10. In some instances, distal holding member 136 includes a single bristle. In other instances, distal holding member 136 may include a pair of bristles disposed on opposite sides of sheath 112. In still other instances, one or more bristles may be positioned along an exterior portion and/or the distal end of sheath 112.

Bristles 136a/136b may be positioned at the distal end of sheath 112. Alternatively, bristles 136a/136b may be positioned a distance proximal of the distal end of sheath 112. In some of these instances, the distal portion of sheath 112 extending distally from bristles 136a/136b may be transparent. This may aid in visualizing the anatomy and/or target tissue during an intervention.

The materials that can be used for the various components of polypectomy device 10 (and/or other polypectomy devices disclosed herein) and the various tubular members disclosed herein may include those commonly associated with medical devices. For simplicity purposes, the following discussion makes reference to sheath 12 and other components of device 10. However, this is not intended to limit the devices and methods described herein, as the discussion may be applied to other similar tubular members and/or components of tubular members or devices disclosed herein.

Sheath 12 and/or other components of device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 10 to achieve the same result.

In some embodiments, a degree of Magnetic Resonance Imaging (MRI) compatibility is imparted into device 10. For example, device 10, or portions thereof, may be made of a material that does not substantially distort the image and create substantial artifacts (e.g., gaps in the image). Certain ferromagnetic materials, for example, may not be suitable because they may create artifacts in an MRI image. Device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A polypectomy device, comprising:
an elongate sheath having a proximal end and a distal end;
a handle assembly coupled to the proximal end, the handle assembly including a first actuator and a second actuator;
a first shaft extending through the sheath and coupled to the first actuator;
an end effector coupled to the first shaft;
a second shaft extending through the sheath and coupled to the second actuator;
a retention member coupled to the second shaft;
wherein the first actuator is designed to shift the end effector between a housed configuration and a deployed configuration; and
wherein the second actuator is designed to shift the retention member between a retracted configuration and a holding configuration.

2. The polypectomy device of claim 1, wherein the first actuator and the second actuator are independently controllable.

3. The polypectomy device of claim 1, wherein the end effector includes a snare loop.

4. The polypectomy device of claim 1, wherein the retention member includes a retention barb.

5. The polypectomy device of claim 1, wherein the retention member includes a spike.

6. The polypectomy device of claim 1, wherein the first shaft and the second shaft are arranged substantially parallel to one another within at least a portion of the sheath.

7. The polypectomy device of claim 1, wherein the sheath includes a distal holding member.

8. The polypectomy device of claim 7, wherein the distal holding member is positioned along an interior surface of the sheath.

9. The polypectomy device of claim 7, wherein the distal holding member includes a bristle.

10. The polypectomy device of claim 7, wherein the distal holding member includes a pair of bristles disposed along opposite sides of the sheath.

11. A method for removing polyps, the method comprising:
advancing a polypectomy snare device through a body lumen to a position adjacent to a polyp, the polypectomy snare device comprising:
an elongate sheath having a proximal end and a distal end,
a handle assembly coupled to the proximal end, the handle assembly including a first handle and a second handle,
a first shaft extending through the sheath and coupled to the first handle;
a snare loop coupled to the first shaft,
a second shaft extending through the sheath and coupled to the second handle,
a retention member coupled to the second shaft,
wherein the first handle is designed to shift the snare loop between a housed configuration and a deployed configuration, and
wherein the second handle is designed to shift the retention member between a retracted configuration and a holding configuration;
actuating the first handle such that the snare loop engages the polyp;
actuating the second handle such that the retention member engages the polyp; and
severing the polyp with the snare loop.

12. The method of claim 11, wherein the first actuator and the second actuator are independently controllable.

13. The method of claim 11, wherein the retention member includes a retention barb.

14. The method of claim 11, further comprising retracting the severed polyp into the sheath.

15. The method of claim 14, wherein the sheath includes one or more bristles positioned along an interior surface of the sheath.

16. The method of claim 15, wherein retracting the severed polyp into the sheath includes engaging the severed polyp with at least one of the one or more bristles.

17. The method of claim 11, further comprising:
actuating the first handle so that the snare loop engages a second polyp;
actuating the second handle such that the retention member engages the second polyp; and
severing the second polyp with the snare loop.

18. A polypectomy snare device, comprising:
an elongate sheath having a proximal end and a distal end;
a handle assembly coupled to the proximal end, the handle assembly including a first handle and a second handle;
wherein the first handle and the second handle are independently controllable;
a first shaft slidably disposed within the sheath and coupled to the first handle;
a snare loop coupled to the first shaft;
a second shaft slidably disposed within the sheath and coupled to the second handle;
a barbed retention member coupled to the second shaft;
wherein the first handle is designed to shift the first shaft axially relative to the sheath; and
wherein the second handle is designed to shift the second shaft axially relative to the sheath.

19. The polypectomy snare device of claim 18, wherein the sheath includes one or more bristles positioned along an interior surface of the sheath.

20. The polypectomy snare device of claim 19, wherein the sheath includes a pair of bristles disposed along opposite sides of the sheath.
